(19) United States
(12) Patent Application Publication
(10) Pub. No.: US 2022/0160789 A1
(43) Pub. Date: May 26, 2022

(54) DUAL ANTIGEN-RECOGNIZING iPS CELL-DERIVED CHIMERIC ANTIGEN RECEPTOR-T-CELL THERAPY

(71) Applicants: JUNTENDO EDUCATIONAL FOUNDATION, Bunkyo-ku (JP); THE UNIVERSITY OF TOKYO, Bunkyo-ku (JP)

(72) Inventors: Miki ANDO, Bunkyo-ku (JP); Norio KOMATSU, Bunkyo-ku (JP); Jun ANDO, Bunkyo-ku (JP); Sakiko HARADA, Bunkyo-ku (JP); Hiromitsu NAKAUCHI, Bunkyo-ku (JP); Tomoyuki YAMAGUCHI, Bunkyo-ku (JP); Motoo WATANABE, Bunkyo-ku (JP)

(73) Assignees: JUNTENDO EDUCATIONAL FOUNDATION, Bunkyo-ku (JP); THE UNIVERSITY OF TOKYO, Bunkyo-ku (JP)

(21) Appl. No.: 17/600,931

(22) PCT Filed: Apr. 1, 2020
(86) PCT No.: PCT/JP2020/015067

§ 371 Date: Oct. 1, 2021

(30) Foreign Application Priority Data
Apr. 2, 2019 (JP) 2019-070808

(51) Int. Cl. A61K 35/545; C07K 14/725; C12N 5/074; A61P 35/00

(52) U.S. Cl. CPC A61K 35/545; A61P 35/00; C12N 5/0696; C07K 14/7051

(57) ABSTRACT

Provided is an immune cell therapy which uses an iPS technology allowing long-term survival in a living body and which exhibits an excellent antitumor effect by recognition of two antigens.

An iPS cell derived from an antigen-specific cytotoxic T cell having a chimeric antigen receptor introduced therein.

### TECHNICAL FIELD

The present invention relates to a novel chimeric antigen receptor-T cell (CAR-T cell) and a medicament using the same.

### BACKGROUND ART

Chimeric antigen receptor-T cells (CAR-T cells) are T cells into which a chimeric antigen receptor has been introduced, and are receiving attention as groundbreaking therapeutic drugs for cancer. The CAR-T cell therapy, a currently approved medicament, is an autologous CAR-T cell therapy in which a chimeric antigen receptor is introduced into T cells induced from the peripheral blood of a cancer patient to produce CAR-T cells and the CAR-T cells are administered to the patient, and has the problem of being extremely expensive. On the other hand, although it is still in the clinical trial stage, an allogeneic CAR-T cell therapy has also been developed, which has the advantage of reducing production cost, but has the problem of reducing the antitumor effect due to immune rejection.

On the other hand, a technique has been developed, which establishes T-iPS cells from antigen-specific cytotoxic T cells (CTLs) and induces the cells to differentiate into functionally rejuvenated CTLs while maintaining their antigen specificity (Patent Literature 1 and Non Patent Literature 1).

### CITATION LIST

### Patent Literature

Patent Literature 1: JP-B-6164746

### Non Patent Literature

Non Patent Literature 1: Experimental Hematology 2017, 47; 2-12

### SUMMARY OF INVENTION

### Technical Problem

However, the anti-cancer effect of the CTL-iPS cell or CAR-T cell therapy is still insufficient depending on the antigen or the type of cancer. Thus, there is a demand for a therapy using immune cells which proliferate more efficiently, exhibit a better antitumor effect, and can survive for a long time in a living body.

### Solution to Problem

Therefore, the present inventors have found that when antigen-specific cytotoxic T cells (CTL) are used as a raw material to produce iPS cells, and then a chimeric antigen receptor (CAR) is introduced to differentiate them, iPS cells having a synergistically enhanced antitumor effect and surviving for a long time in a living body, especially by recognizing two antigens due to iPS cell-derived CTL and CAR, can be obtained, and have completed the invention.

That is, the present invention provides the following [1] to [8].

[1] An iPS cell derived from an antigen-specific cytotoxic T cell (CTL) having a chimeric antigen receptor (CAR) introduced therein.
[2] The iPS cell according to [1], wherein the antigen of the antigen-specific cytotoxic T cell is a viral antigen or a tumor antigen.
[3] The iPS cell according to [1] or [2], wherein the chimeric antigen receptor is a CAR which recognizes a tumor surface antigen.
[4] A medicament comprising the iPS cell according to any one of [1] to [3] as an active ingredient.
[5] A method for producing iPS cells derived from antigen-specific cytotoxic T cells (CTLs) having a chimeric antigen receptor (CAR) introduced therein, the method comprising: introducing Oct3/4, Sox2, Klf4, c-Myc and SV40 large T antigen genes into tumor antigen or viral antigen-specific cytotoxic T cells (CTLs) to obtain antigen-specific cytotoxic T cell (CTL)-derived iPS cells (T-iPSCs); and introducing a chimeric antigen receptor (CAR) into the obtained T-iPSCs to induce their differentiation.
[6] An iPS cell derived from an antigen-specific cytotoxic T cell (CTL) having a chimeric antigen receptor (CAR) introduced therein, for use in treatment of cancer or viral infectious diseases.
[7] Use of an iPS cell derived from an antigen-specific cytotoxic T cell (CTL) having a chimeric antigen receptor (CAR) introduced therein, for producing a medicament for treating cancer or viral infectious diseases.
[8] A method for treating cancer or viral diseases, comprising administering, to a patient in need thereof, iPS cells derived from antigen-specific cytotoxic T cells (CTLs) having a chimeric antigen receptor (CAR) introduced therein.

### Advantageous Effects of Invention

The CTL-derived iPS cells into which CAR has been introduced of the present invention are efficient at introducing a CAR gene with an efficiency of about 100%, have an extremely high antitumor effect resulting from the synergistic effect of the antigen-specific cytotoxicity of CTL and the antitumor effect of CAR, and survive in a living body for a long time. Therefore, they are useful as a therapeutic drug for cancer and viral infectious diseases.

### BRIEF DESCRIPTION OF DRAWINGS

FIG. 1 is a diagram showing a tetramer analysis from induced CTLs to established CTL clones.

FIG. 2 is a diagram showing a tetramer analysis from induced CTLs to established CTL clones.

FIG. 3 is a diagram showing the structure of the vector for LMP1-CAR.

FIG. 4 is a diagram showing the LMP1-CAR expression rate of LMP1-CAR-LMP2-T-iPS.

FIG. 5 is a diagram showing the LMP1-CAR expression rate of LMP1-CAR/LMP2-rejT.

FIG. 6 shows the cytotoxicity of LMP1-CAR/LMP2-rejT and the cytotoxicity of peripheral blood-derived LMP1-CAR to EB virus-related lymphoma cell lines.

FIG. 7 is a diagram showing the tumor growth inhibitory effect of LMP1-CAR/LMP2-rejT on EB virus-related lymphoma cell lines.

FIG. 8 is a diagram comparing the survival rates of LMP1-CAR/LMP2-rejT and LMP2-rejT by using a mouse model.

FIG. 9 is a diagram showing the rate of apoptosis of LMP1-CAR/LMP2-rejT by CID administration.

### DESCRIPTION OF EMBODIMENTS

The cells of the present invention are virus-specific or tumor antigen-specific CTL-derived iPS cells into which CAR has been introduced. The iPS cells are obtained by artificially reprogramming CTLs to obtain TCR-containing iPS cells, and then introducing CAR into these for differentiation. More specifically, the iPS cells are obtained by introducing Oct3/4, Sox2, Klf4, c-Myc and SV40 large T antigen genes into CTL clones to obtain CTL-derived iPS cells, and introducing CAR into the obtained T-iPS cells for differentiation.

The CTL used as a raw material is a T cell which produces cytotoxicity due to having antigen specificity. In particular, viruses that have already infected many adults are excellent as CTLs. Examples thereof include the LMP1 antigen, LMP2 antigen, and EBNA antigen of Epstein Barr Virus, adenovirus antigens such as the penton antigen and the hexone antigen, the CMVpp65 antigen, IE1 antigen, and IE2 antigen of cytomegalovirus, the IE61 antigen, IE62 antigen, IE63 antigen, and ORF10 antigen of varicella-zoster virus (VZV), and herpes simplex virus (HSV) antigens. In addition, as viruses relating to virus-related tumors, virus-specific CTLs such as the E6 antigen and E7 antigen of human papillomavirus (HPV), and the Tax antigen of HTLV-1 virus are also excellent for producing dual antigen-targeting iPSC-derived CAR-rejT (rejuvenated CTL). However, they can also be prepared from CTLs specific to not only viral antigens but also to other antigens, for example, tumor antigens such as the WT1 antigen, NY-ESO-1 antigen, MAGE-1 antigen, and MART-1 antigen.

The CTLs used in the present invention are preferably human CTLs. The human who is the source of these T cells may be a healthy individual or a human suffering from a viral infection. In addition, a preferable human as the source of T cells is a human having the same HLA class as a patient to whom the CAR-containing iPS cells produced according to the present invention should be administered. Furthermore, the same person as the human to whom the CAR-containing iPS cells are administered is preferable. When using allogeneic cells, CTLs induced from human iPS cells whose HLA has been genome-edited can also be used as long as they have an HLA matching the HLA restriction of CTL epitope. The virus is preferably, but not limited to, EB virus and cytomegalovirus, which infect most adults.

Such CTLs can be isolated, for example, from human tissues by a publicly known technique. The human tissues are tissues containing the T cells, and examples thereof include peripheral blood, lymph nodes, bone marrow, thymus, spleen, umbilical cord blood, and lesion tissue. Among these, peripheral blood is preferable from the viewpoint of having a low invasiveness to humans and being easy to prepare. Examples of publicly known techniques for isolating human T cells include magnetic selection using magnetic beads for cell separation, flow cytometry using antibodies against cell surface markers such as CD4 or CD8, and a cell sorter, and the activated T cell induction method using anti-CD3 antibodies and anti-CD28 antibodies. When isolating from human tissues containing T cells having antigen specificity, the T cells having a desired antigen specificity can be purified from human tissue by using multimerized MHC (major histocompatibility complex) (for example, “MHC tetramer” and “Pro 5 (registered trademark) MHC class I pentamer”) to which the desired antigen is bound.

The genes used to reprogram CTLs are Oct3/4, Sox2, Klf4, c-Myc and SV40 large T antigen.

In the present invention, the method for introducing the genes into CTLs is not particularly limited, and a publicly known technique can be appropriately selected and used. For example, when introducing the genes into the CTLs in the form of the nucleic acid encoding the genes, it is possible to insert the nucleic acid encoding the gene (for example, cDNA and RNA) into an appropriate expression vector containing a promoter functioning in T cells, and then introducing the expression vector into the cells by genome editing such as infection, lipofection method, the liposome method, electroporation method, calcium phosphate co-precipitation method, the DEAE-dextran method, microinjection method, electroporation method, and CRISPR/Cas9.

Among such expression vectors, it is more preferable to use a stealth RNA expression vector containing the genes from the viewpoint of reducing the risk of canceration and the introduction efficiency.

A stealth RNA expression vector is a vector designed to prevent the vector from entering the chromosomes and to express the gene continuously and stably in the cytoplasm rather than in the nucleus. The vector can introduce a large gene of 13,000 base pairs or more or also introduce 10 genes simultaneously. It does not damage cells and can be removed when the transgene is not needed, and it has stealth properties in that cells are unable to recognize the vector as a foreign substance.

Such stealth RNA expression vectors include, for example, complexes which do not activate the innate immune structure and consist of negative-sense single-strand RNA (A) containing the following RNA sequences (1) to (8), a single-strand RNA-binding protein (B), and RNA-dependent RNA synthetase:

(1) an RNA sequence for the genes;
(2) a human mRNA-derived RNA sequence constituting a non-coding region;
(3) a transcription initiation signal sequence recognized by the RNA-dependent RNA synthetase;
(4) a transcription termination signal sequence recognized by the RNA-dependent RNA synthetase;
(5) an RNA sequence containing a replication origin recognized by the RNA-dependent RNA synthetase;
(6) an RNA sequence which encodes the RNA-dependent RNA synthetase;
(7) an RNA sequence which encodes a protein for regulating the activity of the RNA-dependent RNA synthetase; and
(8) an RNA sequence which encodes the single-strand RNA binding protein.

In addition, when establishing T-iPS cells, the CTLs are preferably activated by stimulation with anti-CD3 antibodies and anti-CD28 antibodies in the presence of interleukin-2 (IL-2), interleukin-7 (IL-7) or interleukin-15 (IL-15) before introducing the genes, and may be activated by stimulation with at least one substance selected from the group consisting of phytohemagglutinin (PHA), interleukin-2 (IL-2), allogeneic antigen-expressing cells, anti-CD3 antibodies, anti-CD28 antibodies, CD3 agonists and CD28 agonists. Such stimulation can be performed, for example, by adding PHA, IL-2, anti-CD3 antibodies and/or anti-CD28 antibodies and the like to a culture medium and culturing the T cells for a certain period of time. In addition, the anti-CD3 antibodies and/or the anti-CD28 antibodies may have magnetic beads or the like bound thereto, and instead of adding these antibodies to the culture medium, a stimulus may be applied by culturing the CTLs for a certain period of time on a culture dish on which anti-CD3 antibodies and/or anti-CD28 antibodies are bound on the surface. Furthermore, a stimulus may be applied by adding an antigen peptide recognized by the CTLs to the medium together with feeder cells.

In order to apply such stimulus to the CTLs, the concentration of PHA added to the medium is not particularly limited, but is preferably from 1 to 100 μg/mL. Moreover, the concentration of IL-2 added to the medium is not particularly limited, but is preferably from 1 to 200 ng/mL. Furthermore, the concentration of the anti-CD3 antibodies and/or anti-CD28 antibodies added to the medium is not particularly limited, but is preferably from 1 to 10 times the culture amount of the CTLs. In order to apply such stimulus to the CTLs, the concentration of the anti-CD3 antibodies and/or the anti-CD28 antibodies bound on the surface of the culture dish is not particularly limited, but the concentration upon coating is preferably from 0.1 to 100 μg/mL, preferably from 1 to 100 μg/mL for anti-CD3 antibodies, and preferably from 0.1 to 10 μg/mL for anti-CD28 antibodies.

The culture period for such stimulation is not particularly limited as long as it is a period sufficient to apply such stimulus to the CTLs and a period in which the CTLs can be grown to the number of cells required for introducing the genes, but it is usually from 2 to 7 days, and preferably from 3 to 5 days from the viewpoint of gene introduction efficiency, it is. From the viewpoint of infecting by mixing T cells and a vector in a 15 mL tube or increasing the gene introduction efficiency, it is preferable to culture on a culture dish coated with RetroNectin.

As a culture medium for culturing the CTLs and adding PHA, IL-2, anti-CD3 antibodies and/or anti-CD28 antibodies, for example, a publicly known medium suitable for culturing CTLs (more specifically, a Roswell Park Memorial Laboratory (RPMI) 1640 medium, an AIM V™ medium, and NS-A2 containing other cytokines and human serum) can be used. In addition to PHA, IL-2, anti-CD3 antibodies and/or anti-CD28 antibodies, amino acids (for example, L-glutamine) and antibiotics (for example, streptomycin and penicillin) necessary for culturing may be added to the medium. It is also preferable to add IL-7 and IL-15 to the medium instead of IL-2. The concentration of IL-7 and IL-15 added is not particularly limited, but is preferably from 1 to 100 ng/mL for each.

The conditions when introducing the genes into the CTLs or thereafter are not particularly limited, but it is preferable to culture the CTLs into which the genes are introduced under feeder-free conditions. Examples thereof include a well coated with a solution of iMatrix-511, which is a laminin-511 E8 fragment, or vitronectin or Matrigel. The cells can also be established by culturing under feeder cell conditions, and examples of feeder cells include mouse embryonic fibroblasts (MEFs), STO cells, and SNL cells whose cell division has stopped by exposure to radiation or treatment with antibiotics.

Furthermore, in the process of inducing the CTLs to T-iPS cells, it is preferable to add an iPS cell medium from the following day. Then, it is preferable to replace the medium by half every other day to gradually replace the CTL medium with an iPS medium.

In addition, it is preferable to culture while gradually replacing a publicly known medium suitable for culturing CTLs with a medium suitable for culturing iPS cells to coincide with the transition of the CTLs to iPS cells. As such medium suitable for culturing iPS cells, a publicly known medium can be appropriately selected and used, and for example, in the case of iMatrix coating, StemFit AK03N is desirable, in the case of coating with vitronectin, Essential 8 Medium, and in the case of Matrigel, Dulbecco's Modified Eagle Medium/F12 medium (human iPS Cell medium) containing knockout serum replacement, L-glutamine, non-essential amino acids, 2-mercaptoethanol, b-FGF, and the like on feeder cells such as mTeSR, and MEF cells.

In this way, T-iPS cells can be selected by appropriately selecting a publicly known technique. Examples of such a publicly known technique include a method of selecting by observing the morphology of ES cell/iPS cell-like colonies under a microscope. On the other hand, in the case of T-iPS established from a single cell CTL clone, the properties are often similar, and therefore there is also a method of subculturing all the established colonies as they are without selecting each colony of T-iPS cells.

Confirmation that the cells thus selected are T-iPS cells can be made by, for example, a method of detecting the expression of undifferentiated cell-specific markers (SSEA-4, Tra-1-60, Tra-1-81, and the like) in the selected cells by RT-PCR, a method of confirming by ALP staining, or a method of transplanting the selected cells into mice and observing their teratoma formation. In addition, confirmation that the cells thus selected are derived from the CTLs can be made by detecting the state of TCR gene rearrangement by genomic PCR.

As for the time at which these cells are selected and collected, it is preferable to collect them while observing the growth state of the colony, or in the case where a label such as GFP is contained in the Sendai virus vector, to collect when it can be confirmed with a fluorescence microscope that GFP has disappeared. Roughly, it is from 10 to 40 days, preferably from 14 to 28 days after the genes have been introduced into the T cells. As for the culture environment, unless otherwise specified above, it is preferably conditions of 5% CO₂ and from 35 to 38° C., and more preferably 37° C.

In order to introduce CAR into the obtained CTL-derived iPS cells, there are a method of using a viral vector such as a lentiviral vector, genome editing such as CRISPR/Cas9, a method of using a transposon, and the like.

CAR is a chimeric antigen receptor which has the specificity for surface antigens of tumor cells and the ability to activate T cells. The CAR-T therapy is a therapy in which a nucleic acid encoding this chimeric antigen receptor is introduced into T cells, and the obtained transgenic T cells are grown in vitro then infused.

In the present description, “CAR” refers to a fusion protein containing an extracellular domain binding to an antigen, a transmembrane domain derived from a polypeptide different from the extracellular domain, and at least one intracellular domain. The “chimeric antigen receptor (CAR)” is sometimes referred to as the “chimeric receptor,” “T-body,” or “chimeric immune receptor (CIR).” The “extracellular domain binding to an antigen” refers to any oligopeptide or polypeptide capable of binding to a certain antigen, and the “intracellular domain” means any oligopeptide or polypeptide known to function as a domain which transmits a signal activating or inhibiting a biological process inside the cell.

In the present description, “tumor antigen” means an antigenic biomolecule whose expression is newly observed with the canceration of cells. The detection of tumor antigens, for example, immunological detection, is useful for distinguishing between cancerous cells and their mother cells. The tumor antigen in the present invention includes tumor-specific antigens (antigens existing only in tumor cells and not observed in other normal cells) or tumor-related antigens (antigens also existing in the normal cells of other organs/tissues or xenogeneic normal cells, and antigens expressed during development and differentiation).

In the present description, “single chain antibody (scFv)” means a single chain polypeptide derived from an antibody, which retains the ability to bind to an antigen. Examples thereof include an antibody polypeptide formed by recombinant DNA technology and linking to the Fv regions of the heavy chain (H chain) and light chain (L chain) fragments of immunoglobulins via a spacer sequence. Various methods for producing scFv are publicly known, and examples thereof include the methods described in U.S. Pat. No. 4,694,778, Science, Vol. 242, pp. 423-442 (1988), Nature, Vol. 334, p. 54454 (1989), and Science, Vol. 242, pp. 1038-1041 (1988).

In the present description, “domain” means one region within a polypeptide which is folded into a specific structure independently of the other regions.

A representative CAR structure is composed of a single chain antibody which recognizes surface antigens of tumor cells (single chain variable fragment: scFv), a transmembrane domain, and the intracellular domain of the TCR complex CD3C which activates T cells. A CAR with such a configuration is called first-generation CAR. The gene for the single chain antibody portion is isolated, for example, from a hybridoma which produces monoclonal antibodies recognizing a target antigen. T cells expressing CAR can efficiently kill tumor cells by directly recognizing the surface antigens of the tumor cells regardless of the expression of major histocompatibility complex class I on the tumor cells, and activating the T cells at the same time.

For the purpose of enhancing the ability of first-generation CARs to activate T cells, second-generation CARs have been developed, to which the intracellular domain of CD28, a T cell costimulatory molecule, or CD137 (4-1BB), a tumor necrosis factor (TNF) receptor superfamily is linked. As a further improved version, a third-generation CAR is under development, to which two of the costimulatory molecules CD28, 4-1BB or CD134 (OX40) are linked in tandem (Current Opinion in Immunology, Vol. 21, pp 215-223 (2009). In addition, in order to enable the induction of cell death in CAR-T cells as a safety system at the occurrence of side effects, a CAR incorporating the suicide gene inducible caspase-9 is also useful.

Specifically, as a means for introducing CAR, there are a method of introducing a gene using a viral vector such as lentivirus, a method of using a transposon such as piggy bac, or a method of genome editing using CRISR/Cas9 or the like.

To confirm the cells into which CAR has been introduced, the efficiency of CAR gene introduction is analyzed with flow cytometry by staining the protein L binding to scFv.

The cells into which CAR has been introduced can be converted to CAR-rejT cells into which CAR has been introduced by inducing differentiation. As a means for inducing differentiation, the CAR-T-iPS cells after the introduction of the CAR gene are finely crushed and cultured on feeder cells for two weeks. Then, they are cultured with cytokines on feeder cells expressing the Notch Ligand for another four weeks. After four weeks, the floating cells are identified and the T cell receptor (TCR) is stimulated. The CAR gene introduction efficiency after stimulation and antigen specificity are confirmed by the methods described above. The differentiated CAR-rejTs have a CAR gene introduced therein and retain their original antigen specificity.

For the collection of terminal cells, bead selection or FACS sorting using tetramers is performed on the antigen-specific cells when the number of antigen-specific cells is small. If the CAR gene introduction efficiency is low, protein L staining is performed to sort CAR-positive cells. In addition, the required cells can be selected and amplified through, for example, selection of CD8-positive cells.

The iPS cell-derived CTL cells into which CAR has been introduced and which have been produced by the method of the present invention targets two antigens, and as a result, the escape mechanism of the tumor cells is less working, and the cell-killing effect can be efficiently exhibited. They are useful as a therapeutic drug for various malignant tumors and virus-related tumors.

As therapeutic drugs for malignant tumors, tumor antigen-specific rejT for Survivin, NY-ESO-1, WT-1, MAGE3, and the like can be combined with GD2-CAR, HER2-CAR, NY-ESO-1-CAR, MUC1-CAR, CD19-CAR, and the like.

Examples of therapeutic drugs for virus-related tumors include LMP2-CAR/LMP1-rejT in which LMP2-specific rejT is combined with LMP1-CAR/LMP2-rejT or LMP1-specific rejT with LMP2-CAR for EB virus-related lymphoma, which is an EB virus-related tumor, and nasopharyngeal cancer; CD19-CAR/LMP1-rejT and CD19-CAR/LMP2-rejT for EB virus-positive B-cell lymphoma; and NY-ESO-1/Tax-rejT incorporating NY-ESO-1-CAR into Tax-specific rejT and NY-ESO-1/HBZ-rejT incorporating NY-ESO-1-CAR into HBZ-specific rejT for adult T-cell leukemia (ATL).

In addition, a combination of rejT specific to a virus, which is said to have excellent resistance in a living body, with CAR is also useful. For example, a method of combining LMP2-specific rejT, LMP1-specific rejT, CMV-specific rejT, adenovirus-specific rejT, VZV-specific rejT, HSV-specific rejT, and the like with CAR such as GD2-CAR, HER2-CAR, NY-ESO-1-CAR, MUC1-CAR, and CD19-CAR is also useful.

The immune cell therapeutic drug of the present invention is not particularly limited, but can be preferably administered parenterally, for example, intravenously, intraperitoneally, subcutaneously or intramuscularly, and more preferably intravenously. Alternatively, local administration to the affected area is also possible.

The pharmaceutical composition of the present invention can be prepared by formulating the T-iPS cells produced by the method of the present invention through a publicly known pharmaceutical method. For example, the pharmaceutical composition of the present invention can be mainly used parenterally as a capsule, a liquid, a film coating agent, a suspension, an emulsion, an injection (intravenous injection, drip injection, and the like).

In these formulations, pharmacologically acceptable carriers or media, specifically sterilized water or physiological saline, vegetable oils, solvents, bases, emulsifiers, suspending agents, surfactants, stabilizers, vehicles, preservatives, binders, diluents, isotonic agents, soothing agents, bulking agents, disintegrants, buffers, coating agents, lubricants, colorants, solubilizing agents or other additives and the like can be appropriately used in combination. In addition, the pharmaceutical composition of the present invention may be used in combination with a publicly known pharmaceutical composition, immunostimulator, or the like used for the treatment or prevention of the disease.

When administering the pharmaceutical composition of the present invention, the dose can be appropriately selected according to the age, body weight, symptoms, and health condition of the subject, and the type of composition (medicament, food/drink, and the like).

### EXAMPLES

Hereinafter, the present invention will be further described with reference to examples, but the present invention is not limited to these examples.

### Example 1 Establishment of T-iPS Cells Using a Sendai Virus Vector from Human Papillomavirus (HPV)-Specific CTL Clones

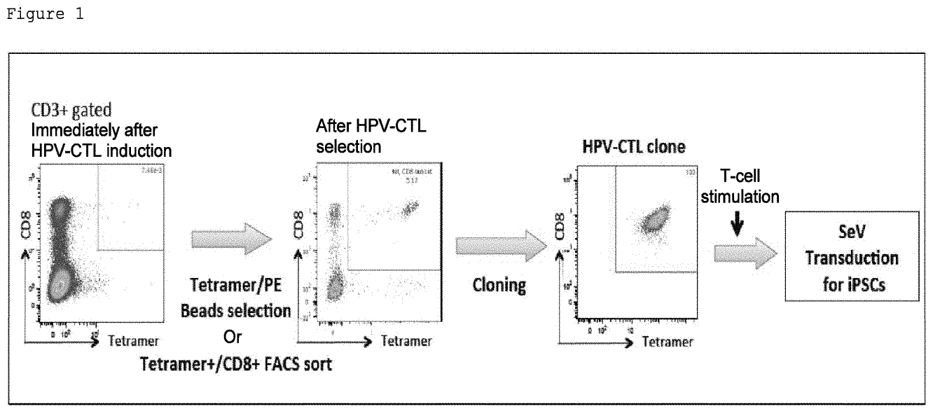

1) Peripheral blood mononuclear cells were isolated from the peripheral blood of a healthy individual, and then dendritic cells were induced for the purpose of antigen presentation. Seven days later, HPV antigen peptides (HPV16-E6 and A2402) were added to the induced dendritic cells and cultured in a CO₂ incubator for 15 minutes, and then co-culture with peripheral blood mononuclear cells was started. Further, co-culture with the dendritic cells was repeated, and after about from 8 to 10 days, the CTLs were stained with MHC tetramer, and then the tetramer positive rate was confirmed by flow cytometry to detect HPV-specific CTLs. After confirming the HPV-specific CTLs, single-cell sorting or tetramer/PE bead selection followed by limiting dilution was performed, and after 50 Gy X-ray irradiation, PBMC, IL2, and PHA were added for stimulation.
2) Tetramer staining of the colonies that had raised after about from 3 to 6 weeks was performed, and the establishment of CTL clones was confirmed by flow cytometry. After confirmation of the establishment, the CTL clones were stimulated with CD3/28, and then the gene was introduced using the following vector A). The CTLs after the gene introduction were transferred to a 6-well plate coated with iMatrix, and culture was started in a CO₂ incubator using a CTL medium supplemented with IL-2 as a medium. (FIG. 1)

### A) SeV4 Factor Vector+SV40 Large T Antigen

3) The day following the introduction of SeV gene, an equal amount of iPS medium (StemFit AK03N) was added, and then half the amount was replaced with StemFit AK03N every other day.
4) After 7 days, colonies of T-iPS cells could be observed, and then colony pick up was performed.

### Example 2 Establishment of T-iPS Cells from Epstein Barr Virus (EBV)-Specific CTL Clones Using a Sendai Virus Vector

1) Peripheral blood mononuclear cells were isolated from the peripheral blood of a healthy individual, and then dendritic cells were induced for the purpose of antigen presentation. Seven days later, EBV LMP2 antigen peptide (A2402) was added to the induced dendritic cells and cultured in a CO₂ incubator for 15 minutes, and then co-culture with peripheral blood mononuclear cells was started. Further, co-culture with the dendritic cells was repeated, and after about from 8 to 10 days, the CTLs were stained with MHC tetramer, and then the tetramer positive rate was confirmed by flow cytometry to detect LMP2-specific CTLs. After confirming LMP2-specific CTLs, single-cell sorting or tetramer/PE bead selection followed by limiting dilution was performed, and after 50 Gy X-ray irradiation, PBMC, IL2, and PHA were added for stimulation.
2) Tetramer staining of the clones that had raised after about from 3 to 6 weeks was performed, and the establishment of LMP2CTL clones was confirmed by flow cytometry. After confirmation of the establishment, the LMP2CTL clones were stimulated with CD3/28, and then the gene was introduced at MOI10 using a Sendai virus vector. The CTLs after the gene introduction were transferred to a 6-well plate coated with iMatrix, and culture was started in a CO₂ incubator using a CTL medium supplemented with IL-2 as a medium.
3) After about 2 weeks, a large number of LMP2CTL-derived T-iPS colonies could be established, and then colony pick up and extended culture was performed. Then, the lentivirus-derived LMP1-CAR vector was infected to prepare LMP1-CAR/LMP2-T-iPS, and then LMP1-CAR/LMP2-rejT were induced for differentiation.
4) The LMP1-CAR expression rate of the differentiated LMP1-CAR/LMP2-rejT was as high as 100%.
5) Tumor cells could be efficiently eliminated by conducting a cytotoxicity test and a co-culture assay.

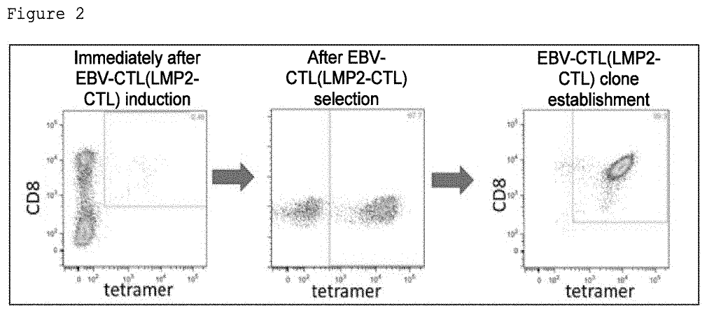

FIG. 2 shows the tetramer staining results for LMP2-CTLs induced from peripheral blood and established CTL clones.

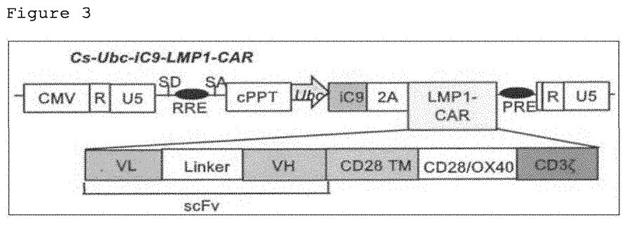

FIG. 3 shows the structure of the vector of iC9-LMP1-CAR-T-iPSCs.

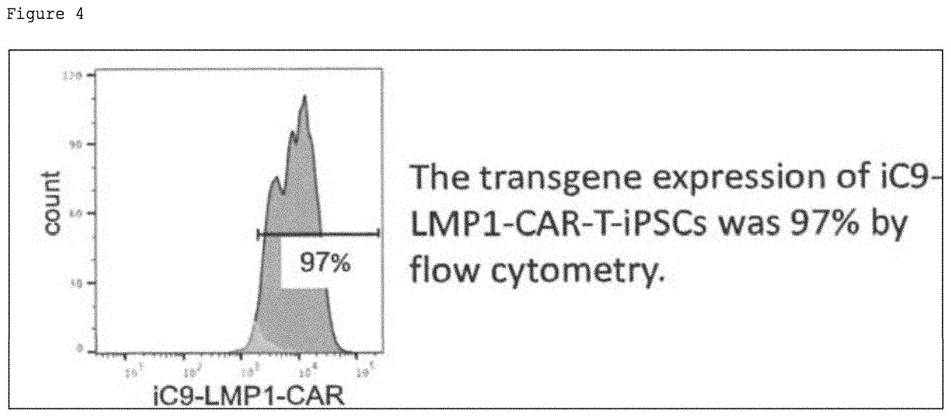

FIG. 4 shows the LMP1-CAR expression rate of LMP1-CAR-LMP2-T-iPS.

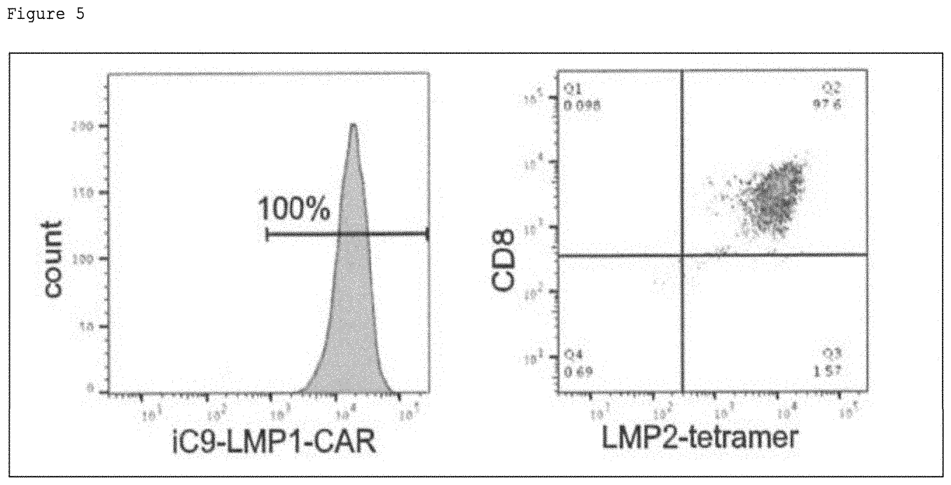

FIG. 5 shows the LMP1-CAR expression rate of LMP1-CAR/LMP2-rejT.

### Example 3

### (Cytotoxicity Test)

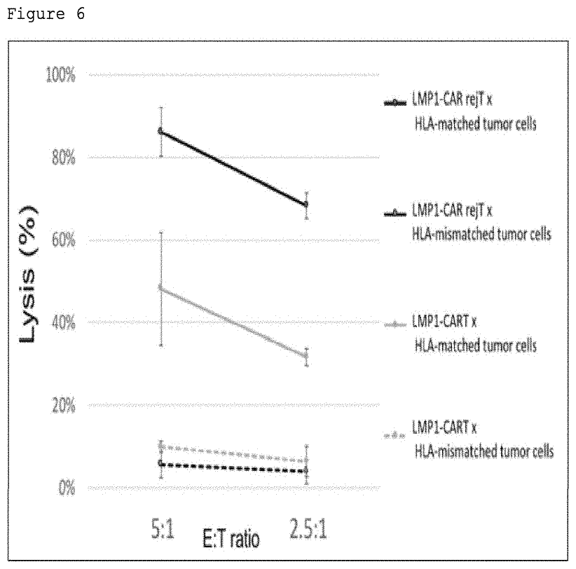

1) A chromium-51 release assay was carried out to compare the cytotoxicity of LMP1-CAR/LMP2-rejT and the cytotoxicity of peripheral blood-derived LMP1-CART to EB virus-related lymphoma cell lines. LMP1-CAR/LMP2-rejT or peripheral blood-derived LMP1-CART as an effector, the chromium-labeled target HLA-matched EB virus-related lymphoma cell line (extranodal NK/T cell lymphoma, nasal type; ENKL) and the control target HLA-mismatched EB virus-infected tumor cell line (LCL) were co-cultured for 6 hours at an effector:target ratio of 5:1 and 2.5:1.
2) After the co-culture, the culture supernatant was transferred to another counter plate, dried, and then measured with a plate reader.
3) LMP1-CAR/LMP2-rejT showed strong antigen-specific cytotoxicity to the HLA-matched EB virus-related lymphoma cell line (70-80%), but showed no cytotoxicity to the control HLA-mismatched tumor cell line, with 10% or less. Peripheral blood-derived LMP1-CART showed a cytotoxicity of about from 30 to 50% to HLA-matched EB virus-related lymphoma cell line. The cytotoxicity of LMP1-CAR/LMP2-rejT was stronger than that of LMP1-CART (FIG. 6).

### Example 4

### (Co-Culture Assay)

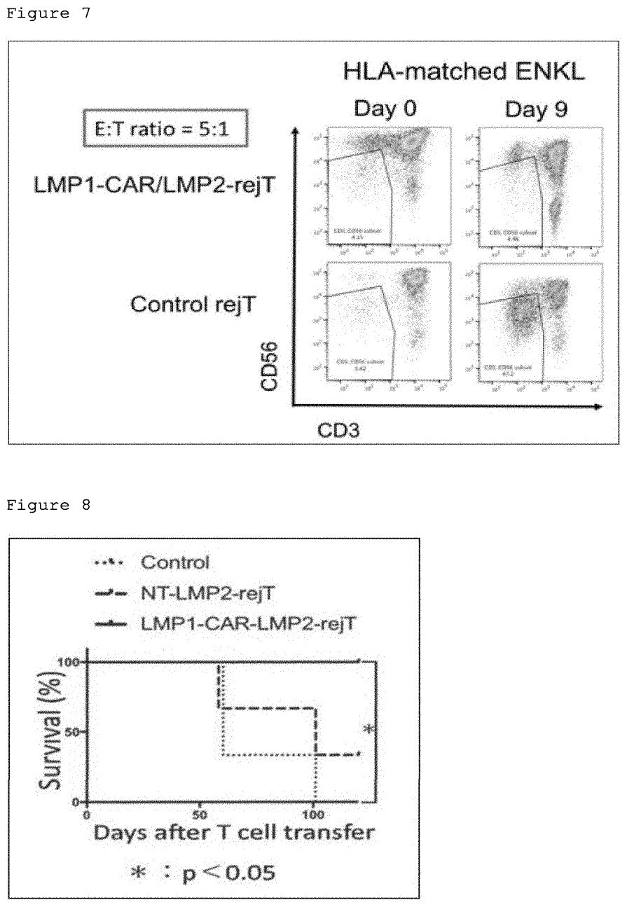

1) A co-culture assay was carried out to compare the cytotoxicity of LMP1-CAR/LMP2-rejT and the cytotoxicity of peripheral blood-derived LMP1-CART to EB virus-related lymphoma cell lines. LMP1-CAR/LMP2-rejT was used as the effector, and rejT specific to antigens other than EB virus was used as the control effector. HLA-matched EB virus-related lymphoma cell line (ENKL) was used as the target. Co-cultured was carried out for 9 days at an effector:target ratio of 5:1.
2) After the co-culture, the ratio of tumor cells and T cells was analyzed by flow cytometry after CD56 and CD3 staining.
3) LMP1-CAR/LMP2-rejT eliminated HLA-matched EB virus-related lymphoma cells on day 9, but the control rejT not having the antigen specificity could not eliminate the tumor cells, and tumor growth was observed (FIG. 7).

### Example 5

### (Anti-Cancer Effect)

Comparative Test of Survival Rates of LMP1-CAR/LMP2-rejT and LMP2-rejT Using Mouse Model

After transplantation of the EB virus-related lymphoma cell line (ENKL) into the abdominal cavity of immunodeficient mice, the survival rate of mice divided into three groups: LMP1-CAR/LMP2-rejT treatment group, LMP2-rejT treatment group, and untreated control were compared.

The results are shown in FIG. 8.

### Example 6

### (Apoptosis by CID Administration)

The Annexin/7-AAD positive rate 48 hours after administration of the low molecular weight compound drug CID, which is a dimerizer, to LMP1-CAR/LMP2-rejT was analyzed to determine the rate of apoptosis.

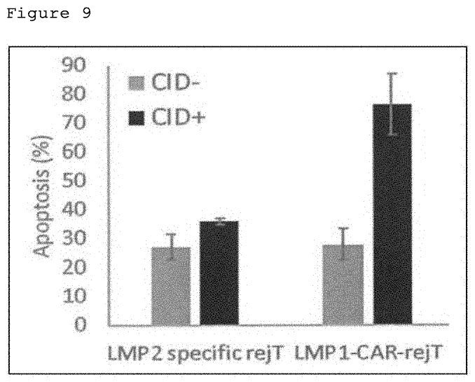

The results are shown in FIG. 9.

1. An iPS cell derived from an antigen-specific cytotoxic T cell having a chimeric antigen receptor introduced therein.

2. The iPS cell according to claim 1, wherein the antigen of the antigen-specific cytotoxic T cell is a viral antigen or a tumor antigen.

3. The iPS cell according to claim 1, wherein the chimeric antigen receptor is a CAR which recognizes a tumor surface antigen.

4. A medicament comprising the iPS cell according to claim 1 as an active ingredient.

5. A method for producing iPS cells derived from antigen-specific cytotoxic T cells (CTLs) having a chimeric antigen receptor (CAR) introduced therein, the method comprising: introducing Oct3/4, Sox2, Klf4, c-Myc and SV40 large T antigen genes into tumor antigen or viral antigen-specific cytotoxic T cells (CTLs) to obtain antigen-specific cytotoxic T cell (CTL)-derived iPS cells (T-iPSCs); and introducing a chimeric antigen receptor (CAR) into the obtained T-iPSCs to induce their differentiation.

6-7. (canceled)

8. A method for treating cancer or viral diseases, the method comprising administering, to a patient in need thereof, iPS cells derived from antigen-specific cytotoxic T cells (CTLs) having a chimeric antigen receptor (CAR) introduced therein.

9. The iPS cell according to claim 2, wherein the chimeric antigen receptor is a CAR which recognizes a tumor surface antigen.

10. A medicament comprising the iPS cell according to claim 2 as an active ingredient.

11. A medicament comprising the iPS cell according to claim 3 as an active ingredient.
